(19) United States
(12) Patent Application Publication
(10) Pub. No.: US 2024/0299288 A1
(43) Pub. Date: Sep. 12, 2024

(54) ACTIVE INGREDIENT COMPRISING OLIGO-GLUCANS DERIVED FROM THE CYTOSOLIC FRACTION OF SACCHAROMYCES CEREVISIAE, AND COSMETIC USES THEREOF

(71) Applicant: SOCIETE INDUSTRIELLE LIMOUSINE D'APPLICATION BIOLOGIQUE, OBJAT (FR)

(72) Inventor: Jean PAUFIQUE, OBJAT (FR)

(21) Appl. No.: 18/294,264

(22) PCT Filed: Aug. 1, 2022
(86) PCT No.: PCT/EP2022/071576

§ 371 Date: Feb. 1, 2024

(30) Foreign Application Priority Data
Aug. 2, 2021 (FR) FR 2108407

(51) Int. Cl. A61K 8/9728; A61Q 19/08

(52) U.S. Cl. CPC A61K 8/9728; A61Q 19/08; A61K 2800/78

(57) ABSTRACT

The present invention relates to an active ingredient comprising oligo-glucans, obtained from the cytosolic fraction of a yeast of the species Saccharomyces cerevisiae, a cosmetic composition comprising said active ingredient and cosmetic uses thereof, in particular to improve the regeneration of the skin and combat aging thereof.

### TECHNICAL FIELD

The invention relates to an active ingredient comprising oligo-glucans, obtained from the cytosolic fraction of a yeast of the species Saccharomyces cerevisiae, a cosmetic composition comprising said active ingredient and cosmetic uses thereof, in particular to improve the regeneration of the skin and combat skin aging.

### PRIOR ART

The regeneration process, for example for the skin, is a dynamic and complex process implementing numerous cellular actors, during which new tissue develops to restore previously damaged areas and thus ensure its healing.

In the skin tissue, three systems are involved in the tissue regeneration process: the skin, vascular and immune system.

The skin tissue is currently recognized as a complex organ comprising in particular fibroblasts, which play a pivotal role between these three systems to act on epidermal renewal and matrix remodeling, but also on vascularization, thus allowing regeneration of damaged tissue.

In the skin system, the renewal of the epidermis relies on keratinocytes, which stop adhering to the basal membrane to migrate, proliferate and differentiate into functional corneocytes. In the dermis, matrix remodeling is carried out by the fibroblasts, after they have migrated and are differentiated into myofibroblasts.

The vascular system makes it possible inter alia to transport nutrients, mediators and cells, which are essential for skin regeneration. To do this, endothelial cells migrate to group together in a cohesive manner, in order to form an organized and regular vascular network.

Finally, in the immune system, there is a population of macrophages, called pro-regenerating macrophages, which are capable of interacting with the fibroblasts making it possible to stimulate the matrix remodeling and to promote vascularization.

During the regeneration process, the fibroblasts secrete growth factors which will act on these systems by activating the cell proliferation, migration and differentiation pathways. These factors act in a paracrine manner on the metabolism of the surrounding fibroblasts, but also on the metabolism of the other cell types present in skin tissue.

However, during aging, cell metabolism slows down. The capacity of the fibroblasts to secrete growth factors to interact with their environment decreases. The endogenous regeneration process then slows down and the successive damage that our skin undergoes is no longer correctly repaired. The quality of the epidermal renewal decreases, the dermal matrix sags and the vascular network becomes disorganized.

The first signs of skin aging then appear. The complexion becomes dull, loses uniformity, the skin is more prone to redness and wrinkles become more pronounced. However, the radiance of the complexion as well as wrinkles reflect skin health. A dull complexion gives a tired appearance and can accentuate the signs of age, which may have a significant impact on self-esteem and the image that one conveys.

Many cosmetic products aim to combat the signs of skin aging, for example to combat wrinkles, improve skin hydration, or improve complexion radiance. However, these cosmetic ingredients act on one of the systems and do not seek comprehensive action over the entire regeneration process.

The exogenous supply of growth factors in the context of tissue regeneration has certainly demonstrated its efficacy. However, this solution is based on regenerative medicine techniques which are judged to be very invasive. Therefore, it cannot be part of a beauty routine.

Another rejuvenation technique called PRP (platelet-rich plasma) consists in intradermal injection of an autologous concentrate of growth factors present in the PRP, previously obtained from a blood sample of the patient. Here again, this technique is controversial due to its invasiveness, and its efficacy is limited by the initial concentration of the patient in PRP.

Facilitating skin regeneration processes while minimizing the impact for the patient remains a major issue for industries in the dermatological and cosmetic sector.

The inventor has thus studied the metabolism of older fibroblasts, but also the metabolism of keratinocytes, endothelial cells and macrophages in the presence of the secretome of older fibroblasts and has observed:
slowing down the migration of the fibroblasts and their differentiation into myofibroblasts, leading to a reduction in the synthesis of the collagen I network;
decreasing keratinocyte migration, which is a key step of the renewal of the epidermis,
a reduction in the pro-regenerating immune population, which is involved in matrix remodeling;
a reduction in endothelial cell metabolism, leading to a defect in the formation of the vascular network.

The metabolism of the skin, immune and vascular system is therefore reduced, not allowing optimal regeneration.

For this purpose, the inventor has identified a specific active agent obtained from a cytosolic fraction of the yeast of Saccharomyces cerevisiae making it possible to act on the entire process of skin regeneration.

Granted, cosmetic active ingredients obtained from Saccharomyces cerevisiae are already known. Mention may in particular be made of an anti-aging solution containing a culture lysate of S. cerevisiae for stimulating skin cell biosynthesis (U.S. Pat. No. 5,019,391).

Other anti-aging and moisturizing cosmetic solutions rely on yeast hydrolysates, in particular yeast hydrolysates of yeast of S. cerevisiae. Mention may thus be made of a cosmetic composition comprising mannoproteins derived from the cell wall of yeasts such as Saccharomyces described in EP2567688. Said extract is obtained from chemical, enzymatic or physicochemical hydrolysis of the yeast wall.

Application WO 2009/101503 also discloses the use of yeast hydrolysate of S. cerevisiae consisting of proteins derived from the wall of the hydrolyzed yeasts, the molecular weight of which is between 1 and 5 kDa.

Thus, the known solutions are particularly aimed at a hydrolysate of S. cerevisiae, and more particularly a hydrolysate obtained from the wall of the yeast of S. cerevisiae. This hydrolysate is rich in proteins, known to combat skin aging.

There is therefore a need for an alternative natural, non-invasive cosmetic solution also making it possible to act on the skin, vascular and immune systems simultaneously in order to stimulate the endogenous capacities of the skin to improve its regeneration capacities.

### SUMMARY OF THE INVENTION

This is the objective of the present invention which, for this purpose, proposes a novel active ingredient obtained from the cytosolic fraction of the yeast of Saccharomyces cerevisiae.

Yeast is a eukaryotic microorganism, composed of a cell wall surrounding the plasma membrane. This wall consists of an outer layer of mannoproteins associated with glucans and an inner layer of glucans associated with chitins. The cytoplasmic membrane is composed mainly of phospholipids and protein complexes. The content of the cell, including the nucleus and the cytoplasmic organelles, is included in a liquid phase called cytosol.

The yeast of Saccharomyces cerevisiae is a single-cell eukaryote well known to a person skilled in the art, which is in the form of isolated, ovoid to rounded cells. It has been used since ancient times to manufacture fermented beverages. However, it was isolated and identified beginning in the middle of XIX^{th} century to replace the traditional bakers' leaven through its use in a more reliable and faster fermentation process than leaven.

Uses as a medicament for certain gastrointestinal diseases have also been reported, but also as cosmetic active agent, as mentioned above.

However, the inventor surprisingly discovered the effects associated with an extract specifically obtained from the cytosolic fraction of a yeast of Saccharomyces cerevisiae. Said extract comprising oligo-glucans with a molar mass of between 2 and 5 kDa.

Thus, the invention relates to an active principle comprising at least one extract of Saccharomyces cerevisiae, said extract corresponding to the cytosolic fraction comprising oligo-glucans with a molar mass of between 2 and 5 kDa.

Preferentially, said extract consists of the cytosolic fraction of a yeast of Saccharomyces cerevisiae. More preferentially, the extract is a cytosolic fraction obtained by a mechanical lysate of Saccharomyces cerevisiae.

The invention also relates to a method for preparing the active ingredient according to the invention, comprising in particular solubilizing Saccharomyces cerevisiae in water by mechanical homogenization, that is, a mechanical lysate making it possible to break the membranes of the yeasts to extract the cytosolic fraction, separating the soluble and insoluble phases, ultrafiltration followed by the recovery of the filtrate, and molecular sorting by ion chromatography or membrane filtration.

The active ingredient according to the invention is of natural origin and advantageously shows an improvement in skin regeneration. To achieve this, the active ingredient according to the invention promotes interconnectivity of the skin, vascular and immune systems. It acts on these three systems, which makes it possible to combat skin aging by providing a comprehensive solution. Thus, the active ingredient makes it possible to reduce under-eye circles and wrinkles and to improve the radiance of the complexion. It thus addresses the problems of the prior art.

The active ingredient according to the invention can be used for cosmetic applications, preferentially when it is integrated into a cosmetic composition, preferentially a composition in a form suitable for topical application.

The invention therefore also relates to a cosmetic use of a composition comprising at least 0.1 wt. % of the active ingredient according to the invention, in particular to improve skin regeneration. Preferentially, the invention improves the interconnectivity of the skin, vascular and immune systems.

Other features and advantages will emerge from the detailed description of the invention and the following examples.

### BRIEF DESCRIPTION OF THE FIGURES

FIG. 1 shows the effect of the active ingredient according to the invention on the skin regeneration capacity, after 14 days of a twice-daily application.

FIG. 2 shows the effect of the active ingredient according to the invention on the biomechanical properties of the skin after 21 and 42 days of twice-daily treatment.

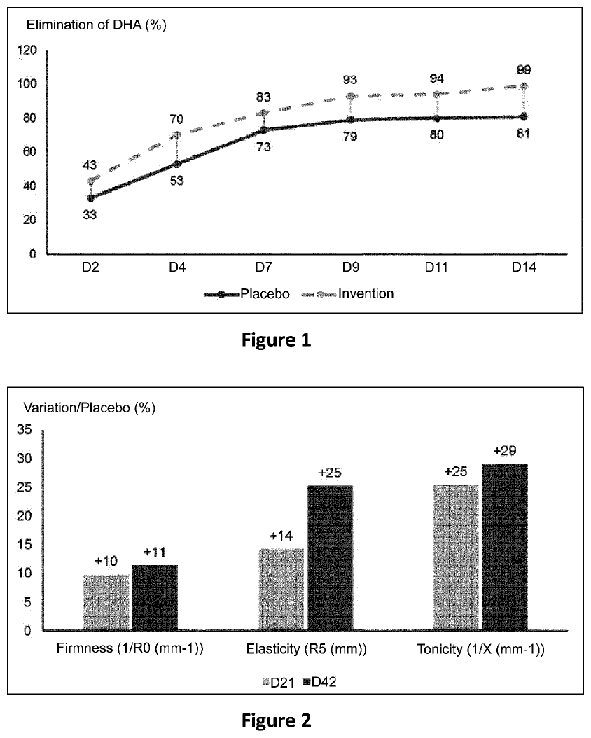

### DETAILED DESCRIPTION OF THE INVENTION

### Definition

Within the meaning of the invention, “cosmetic active ingredient” means an extract comprising at least one molecule, preferentially a set of molecules having a cosmetic effect on the skin. Preferentially, the cosmetic effect on the skin is an anti-aging effect, in particular a regenerating effect of the skin.

For the purposes of the invention, the term “regenerating effect” means the capacity of a living being to repair itself, and in particular the ability of the skin to be reconstituted in response to an attack (burn, scratches, cut) or to natural degradation. It is in particular a global effect that acts on the skin, vascular and immune systems by improving the interconnectivity of said systems.

For the purposes of the invention, “interconnectivity” refers to exchanges between the various systems (skin, vascular and immune) inside the skin tissue. In the context of the invention, the fibroblast plays a pivotal role in regeneration by interconnecting the three systems via the secretion of a pool of growth factors.

“System” is understood to mean a set of interdependent elements constituting it. In the context of the invention, mention may be made of the skin, vascular and also immune systems. The vascular system consists of arterioles, capillaries and venules. The skin system is the surface in contact with the outside environment and comprises the epidermis, the dermis and the hypodermis. The immune system consists of several types of immune cells, in particular dendritic cells, T lymphocytes, macrophages and mast cells.

For the purposes of the invention, the term “mechanical lysate” refers to the product resulting from mechanical lysis, that is, the destruction of the membrane of a biological cell by a physical agent, such as a mechanical means, as opposed to a chemical or biological agent, such as temperature or an enzyme or else natural autolysis of the cell.

For the purposes of the invention, “mechanical lysate of Saccharomyces cerevisiae” refers to any active ingredient from the yeast Saccharomyces cerevisiae, obtained by a method comprising at least one step of solubilization of Saccharomyces cerevisiae consisting in mechanical homogenization. Mechanical lysis, that is, the destruction of the membrane of a biological cell, is carried out by a physical technique, such as a mechanical means, as opposed to a chemical or biological agent, such as an enzyme, the temperature or else the natural autolysis of the cell. The term “mechanical lysate” excludes molecules produced only by fermentation of Saccharomyces cerevisiae, but also from enzymatic or chemical hydrolysis.

For the purposes of the invention, “oligo-glucan” refers to a glucan fraction. Glucans and oligo-glucans are polysaccharides consisting exclusively of glucose. These glucose molecules can be linear or branched. In particular, an oligo-glucan is a glucose oligomer of small size, that is, less than or equal to 5 kDa.

### Active Ingredient According to the Invention

The present invention therefore relates to a cosmetic active ingredient comprising at least one yeast extract of the species Saccharomyces cerevisiae which is particularly useful for improving skin regeneration.

The active ingredient according to the invention comprises at least one extract of Saccharomyces cerevisiae, said extract corresponding to the cytosolic fraction comprising oligo-glucans with a molar mass of between 2 and 5 kDa.

Thus, said extract excludes any other part or element of the yeast, such as the wall or the membranes of the yeast. Preferentially, said extract consists of the cytosolic fraction of the yeast of Saccharomyces cerevisiae. Advantageously, the extract is a mechanical lysate of Saccharomyces cerevisiae.

According to a particularly preferred object, said extract is likely to be obtained by a method comprising the following steps:
a. solubilizing at least 50 g/l of Saccharomyces cerevisiae in water, by mechanical homogenization,
b. separating the soluble and insoluble phases,
c. ultrafiltration and recovery of the filtrate, and
d. molecular sorting, in particular by membrane filtration.

Preferentially, the extract is obtained from the soluble phase.

The extract constituting the cosmetic active ingredient according to the invention was characterized by the inventor. The latter comprises oligo-glucans, which preferentially represent between 10 and 35 wt. % dry matter of the extract.

Preferentially, the extract according to the invention consists of the cytosolic fraction of the yeast; the latter does not comprise membranes, and as a result said extract comprises little mannose. Even more preferentially, the extract according to the invention comprises a glucose/mannose ratio of greater than 60%.

Preferably, the extract according to the invention also comprises a protein fraction. The distribution and the quantity of the protein fraction can be determined by assaying the total nitrogen according to the KJELDHAL method (reference: Official method of analysis of the A.O.C., 12th ed. W Horwitz, E. D., New-York, 15-60, 1975). Preferentially, the extract comprises between 45 and 55 wt. % dry material of peptides of the extract according to the KJELDHAL method, of which 96% are peptides having a molecular mass of less than 2000 Da.

The inventor also determined the ash content. The ash content can be determined by weighing the residues from the incineration of the samples of the active ingredient according to the invention at 550° C. in an electric muffle furnace. Preferentially, the active ingredient according to the invention has an ash content of between 10 to 22 wt. % dry material of the active ingredient.

Additionally, the extract according to the invention also comprises between 0 and 31 wt. % dry material of other cell metabolites present in the cytosol, such as organic acids, DNA, etc.

The cosmetic active ingredient according to the invention can be in liquid form, in solid form or in film form.

When the active ingredient according to the invention is in liquid form, it is exclusively constituted by the extract of the cytosolic fraction of the yeast of Saccharomyces cerevisiae, optionally accompanied by stabilizer and/or preservative system.

The extract in liquid form is preferentially in the form of a clear liquid aqueous solution, with a characteristic odor and a light yellow color. However, it may be more colored and/or be discolored by any method known to a person skilled in the art.

The content of dry matter can be determined by weighing the residues resulting from the drying of the extract according to the invention at 105° C. in an oven until a constant weight is obtained. Preferentially, the extract according to the invention in liquid form has a dry matter content of: 10 g/L to 50 g/L, even more preferentially from 15 g/L to 35 g/L.

The active ingredient according to the invention can also be presented in the form of a film. In this case, the extract of the cytosolic fraction of Saccharomyces cerevisiae preferentially represents at least 0.1 wt. % of the film.

When it is in the form of a film, the active ingredient comprises:
a. at least the lysate of the cytosolic fraction of Saccharomyces cerevisiae according to the invention, and
b. at least one mineral filler, and/or
c. at least one polymer of natural origin, and/or
d. at least one plasticizer, and/or
e. at least one surfactant.

The polymer of natural origin may be chosen from: Pectin, tamarind gum, alginate, pullulan, psyllium, xanthan, guar, tara, carob, agar, gum arabic, gellan, dextran, carrageenan, cellulose, konjac and chitosan.

The plasticizer may be chosen from: Glycerol, sorbitol, saccharose, erythritol, urea, propylene glycol and butylene glycol.

The mineral filler may be chosen from: Calcium carbonate, green clay, kaolin, perlite, talc, magnesium silicate, mica, diatomaceous sericite, silica, calcium sulfate, calcium chloride, potassium chloride, iron oxide and zinc oxide. The active ingredient may also comprise a pigment for coloring the film.

When the active ingredient according to the invention is in solid form, it is preferentially constituted by the extract of the cytosolic fraction of the yeast of Saccharomyces cerevisiae as described above and by a support selected from maltodextrin, gum arabic, soy lecithin or isomalt. According to one particularly suitable embodiment, the extract represents at least 10 wt. % of the active ingredient and the support at most 90 wt. % of the active ingredient.

In the case of a solid form wherein the active ingredient is associated with a support, the sugars, protein contents and ash in the active ingredient are modified, the support generally consisting predominantly of sugars.

### Method of Extraction

The extract constituting or contained in the active ingredient according to the invention can be obtained by any process comprising at least one step of extraction of the cytosolic fraction of the yeast from the yeast of Saccharomyces cerevisiae, in particular a step of mechanical lysis.

According to a particularly suitable embodiment, the active ingredient according to the invention is obtained by implementing the following steps:
a. solubilizing at least 50 g/l of Saccharomyces cerevisiae in water, by mechanical homogenization,
b. separating the soluble and insoluble phases,
c. ultrafiltration and recovery of the filtrate, and
d. molecular sorting, in particular by membrane filtration.

Mechanical homogenization is carried out by any means known to a person skilled in the art, for example by mechanical grinding using a homogenizer.

The separation of the soluble and insoluble phases is carried out by any means known to a person skilled in the art, for example by centrifugation, filtration or decantation. Preferentially, the separation of the soluble and insoluble phases is carried out by centrifugation, thus allowing the recovery of the soluble phase containing inter alia the soluble sugar and protein fractions.

The method comprises an ultrafiltration step after the recovery of the soluble phase in order to carry out molecular sorting, for example by ion chromatography or membrane filtration. Thus, this ultrafiltration step allows purification of the recovered soluble phase. It is carried out in order to eliminate high molecular weight molecules (enzymes and polymers, wall elements, membranes, etc.), that is, molecules with a molecular weight greater than 10 kDa.

The lysate obtained at this stage may optionally be further concentrated and/or purified, preferentially by successive ultrafiltration steps through filters with different cutoff thresholds, while retaining the filtrates at each step and/or by a chromatographic method.

The product obtained after lysis and ultrafiltration, before or after concentration and sterilizing filtration, is a lysate of Saccharomyces cerevisiae, and constitutes the first form of the active ingredient according to the invention, being in liquid form at that time.

The lysate obtained can then be dried and associated with a support, in order to be in solid form. This phase can be carried out by implementing the following steps:
a. an atomization support, preferably maltodextrin, is added to the product in liquid form, at most 90% (by mass/volume),
b. this solution is then concentrated under vacuum;
c. any bacteria that may be present are removed by heat treatment or filtration;
d. atomization makes it possible to obtain a powder.

The steps of the methods described above, taken individually are common in the field of extraction of active ingredients from natural raw materials and the person skilled in the art is able to adjust the parameters on the basis of his general knowledge.

### Composition

The active ingredient according to the invention may be integrated into a cosmetic composition, in particular a composition comprising at least 0.1 wt. % of said active ingredient.

Said composition according to the invention preferentially comprises a physiologically acceptable medium, preferentially a cosmetically acceptable medium, that is, which does not cause sensations of discomfort for the user, such as redness, tugging or tingling.

The composition may be in different galenical forms, suitable for topical application to the skin.

These compositions may in particular be in the form of oil-in-water emulsions, water-in-oil emulsions, multiple emulsions (Water/Oil/Water or Oil/Water/Oil) which may optionally be microemulsions or nanoemulsions, or in the form of solutions, suspensions, hydrodispersions, aqueous gels, powders, or foundation. They may be more or less fluid and have the appearance of creams, emulsions, gels, masks or any other aspects of healthy skin care cosmetics.

They can be compositions comprising at least 0.1% of the liquid active ingredient according to the invention, preferentially between 0.5 and 10% or comprising at least 0.01% of a powder active ingredient according to the invention.

As an additive, the compositions according to the invention may contain at least one compound selected from:
a. oils, which may be chosen in particular from linear or cyclic, volatile or non-volatile silicone oils,
b. waxes, such as ozokerite, polyethylene wax, beeswax or carnauba wax,
c. silicone elastomers,
d. surfactants, preferably emulsifiers, whether non-ionic, anionic, cationic or amphoteric,
e. co-surfactants, such as linear fatty alcohols,
f. thickeners and/or gelling agents,
g. humectants, such as polyols such as glycerin,
h. dyes, preservatives, fillers,
I. tighteners,
j. sequestrants,
k. perfumes,
l. and mixtures thereof, without this list being exhaustive.

Examples of such additives are cited in particular in the CTFA Dictionary (International Cosmetic Ingredient Dictionary and Handbook published by the Personal Care Product Council).

Of course, a person skilled in the art will take care to choose any additional, active or non-active compounds, and their quantity, such that the advantageous properties of the mixture are not, or substantially not, altered by the envisaged additive.

These compositions are intended to be used on healthy skin, in particular mature skin, in particular to improve skin regeneration and to obtain an overall anti-aging effect, preferentially an anti-wrinkle effect and/or to improve the radiance of the complexion and/or to reduce under-eye circles.

The invention therefore also relates to a cosmetic method for treating the skin to improve skin regeneration, in particular to improve the complexion radiance and/or to reduce under-eye circles and/or to reduce wrinkles which consists in topical application to the skin of a healthy person of such an active ingredient according to the invention or of such a composition according to the invention.

### Use

The active ingredient according to the invention or the composition according to the invention has characteristics which allow its use in cosmetics and in particular to limit the natural effects related to skin aging, and thus to improve skin regeneration and to obtain an overall anti-aging effect, preferentially to reduce wrinkles and/or to reduce under-eye circles and/or to improve the radiance of the complexion.

To achieve this, the active ingredient or composition according to the invention improves interconnectivity of the skin, vascular and immune systems. Preferentially, the active ingredient or the composition according to the invention increases the synthesis and secretion of the growth factors KGF, EGF, IGF, FGF, PDGF, TGF-β and VEGF.

However, in the skin system, in the epidermis: KGF (keratinocyte growth factor) is known to stimulate keratinocyte migration, which is an essential step for epidermal renewal. Likewise, EGF (epidermal growth factor) promotes epithelial cell proliferation. Finally, IGF (insulin growth factor) promotes keratinocyte proliferation, migration and differentiation. In the dermis, matrix remodeling is induced by four main growth factors. Thus, IGF has a mitogenic role on fibroblasts, FGF-2 or bFGF, member of the FGF (fibroblast growth factor) family promotes fibroblast proliferation and migration. PDGF (platelet-derived growth factor) also stimulates the survival of fibroblasts and their synthesis of collagen. Finally, TGF-β (transforming growth factor) stimulates fibroblast proliferation and induces their differentiation into myofibroblasts.

At the immune system, the TGF-β released by the fibroblasts is capable of activating the resident macrophages into pro-regenerating macrophages. They then secrete numerous growth factors, such as TGF-β.

At the vascular network: VEGF (vascular endothelial growth factor) promotes the formation of new capillaries by the endothelial cells.

By increasing the synthesis and the secretion of KGF, EGF, IGF, FGF, PDGF, TGF-β and VEGF, the active ingredient according to the invention thus makes it possible respectively to stimulate keratinocyte migration, epithelial cell proliferation and keratinocyte differentiation, to promote fibroblast proliferation and migration, to stimulate fibroblast survival and collagen synthesis, to stimulate fibroblast proliferation and induce their differentiation into myofibroblasts, and thus to activate resident macrophages into pro-regenerating macrophages.

As a result, the active ingredient according to the invention acts on:
the skin system: by significantly improving the migration of fibroblasts (+97%) and their differentiation into myofibroblasts, which are essential actors in skin regeneration (α-SMA increased by 40%), and by promoting the synthesis of the collagen 1 network (+53%), the active agent stimulates matrix renewal. This is reflected in vivo by an improvement in the structure of the dermis: the quality of the matrix is increased by 32% and the quality of the fibers is increased by 20%, compared to the placebo. In the epidermis, the active agent also stimulates keratinocyte migration (+99%), which is essential to epidermal dynamics.
the immune system: by promoting the pro-regenerating population (+132%), which is capable of interacting in return with the fibroblasts, it becomes possible to promote matrix remodeling.
the vascular system: by restoring the migration of endothelial cells and their cohesion (+93% and +77%, respectively), the active agent promotes the formation of the vascular network (+55%).

By restoring all of the growth factors, the active ingredient according to the invention thus has an overall anti-aging action. As a result, wrinkles and under-eye circles are significantly reduced, the microvascular network becomes homogeneous, the skin is firmer and the radiance of the complexion is revived. It is particularly suited to healthy mature skin.

The invention is now illustrated by non-limiting examples of compositions according to the invention and by results of tests.

### EXAMPLES

### Example 1: Active Ingredient According to the Invention

The active ingredient is obtained by implementing the following steps:
a. Solubilization of the Saccharomyces cerevisiae yeasts by mechanical homogenization (lysis),
b. Separation of the soluble/insoluble phases,
c. Ultrafiltration,
d. Recovery of the filtrate,
e. Molecular sorting by membrane filtration, intended to select the molecules,
f. Discoloration and deodorization, and
g. Filtration and sterilizing filtration.

The extract constituting the active ingredient according to the invention consists of the cytosolic fraction of the yeasts of S. cerevisiae, the membranes of which are removed, and virtually no mannose remains in said extract (the mannose level of the glycosidic fraction is less than 2%).

The obtained active ingredient has the following analytical characteristics:
a dry matter content: 24.4 g/L,
a protein fraction content according to the Kjeldah method 1:12.4 g/L (50.8%)
a total sugar content: 4 g/L (16.4%)
ash level: 4.3 g/L (17.6%)
cell metabolites, organic acids, DNA, etc. (15.20%).

The glycosidic fraction of the active ingredient obtained is more particularly constituted by 10% of monosaccharides with a molar mass of less than 180 Da and 90% of oligosaccharides with a molar mass of between 360 Da and 5000 Da. More particularly, the glycosidic fraction is composed of about 0.5% of glucosamine, 1.7% of galactose, 1.4% of mannose and 96.4% of glucose. Or a glucose/mannose ratio of 68.9%.

The sugars present in the active ingredient are in the form of oligo-glucans, preferentially with a size of between 2 and 5 kDa.

### Example 2—Active Ingredient Outside the Invention

The active ingredient is obtained by implementing the following steps:
a. Solubilization of Saccharomyces cerevisiae yeasts in a basic aqueous medium at least at 200 g/L,
b. Enzymatic hydrolysis,
c. Separation of soluble and insoluble phases by centrifugation, decantation or filtration,
d. Sterilizing filtration

The obtained active ingredient has the following analytical characteristics:
a dry matter content: 54.6 g/L,
a protein content according to the Lowry method: 23.2 g/L (42%),
a total sugar content: 18.3 g/L (34%),

The active ingredient according to example 2 consists of 34% sugars including 5% disaccharide and 95% saccharides with sizes greater than 1440 Da. And a glucose/mannose ratio of 14.0%.

### Example 3—Active ingredient outside the invention

The active ingredient is obtained by implementing the following steps:
a. Solubilization of Saccharomyces cerevisiae yeasts in a slightly basic aqueous medium at least at 20% optionally with the addition of an alcohol in order to promote the solubilization of the yeasts,
b. hydrolysis of the proteins in a basic medium,
c. stirring from 8 to 52 h,
d. first filtration in order to separate the soluble and insoluble phases,
e. washing against water in order to purify the active fraction,
f. successive concentrations and filtrations, and
g. sterilizing filtration.

The obtained active ingredient has the following analytical characteristics:
a dry matter content: 42.7 g/L,
a protein content according to the Lowry method: 10.4 g/L (24%)
a total sugar content: 25.7 g/L (60%)

The active ingredient according to example 3 is thus predominantly composed of mannans.

### Example 4-Active ingredient outside the invention

The active ingredient is obtained by implementing the following steps:
a. Solubilization of the Saccharomyces cerevisiae yeasts by mechanical homogenization (lysis),
b. Separation of the soluble/insoluble phases,
c. recovering the soluble phase,
d. Molecular sorting by membrane filtration, intended to select the molecules,
e. Discoloration and deodorization, and
f. Filtration and sterilizing filtration.

The obtained active ingredient has the following analytical characteristics:
a dry matter content: 22 g/L,
a protein fraction content according to the Kjeldah method: 12.2 g/L (56%),
a total sugar content: 8.1 g/L (37%),
ash level: 6.4 g/L (17%).

### Example 5—Active Ingredient Outside the Invention

The active ingredient is obtained by implementing the following steps:
a. Solubilization of the Saccharomyces cerevisiae yeasts by mechanical homogenization (lysis),
b. Separation of the soluble/insoluble phases,
c. Ultrafiltration,
d. Recovery of the filtrate,
e. Molecular sorting by membrane filtration, intended to select the molecules,
f. Recovery of the retentate,
g. Discoloration and deodorization, and
h. Filtration and sterilizing filtration.

The obtained active ingredient has the following analytical characteristics:
a dry matter content: 29.7 g/L,
a protein fraction content according to the Kjeldah method: 6.2 g/L (21%),
a total sugar content: 29.5 g/L (99%),
ash level: 7.9 g/L (16%).

The active ingredient obtained is composed of molecules with sizes greater than 5 kDa.

### Example 6—Active ingredient outside the invention

The active ingredient is obtained by implementing the following steps:
a. Solubilization of the Saccharomyces cerevisiae yeasts by mechanical homogenization (lysis),
b. Separation of the soluble/insoluble phases,
c. Ultrafiltration,
d. Recovery of the filtrate,
e. Molecular sorting by membrane filtration, intended to select the molecules,
f. Recovery of the filtrate,
g. Discoloration and deodorization, and
h. Filtration and sterilizing filtration

The obtained active ingredient has the following analytical characteristics:
a dry matter content: 35.6 g/L,
a protein fraction content according to the Lowry method: 9 g/L (25%),
a total sugar content: 9.9 g/L (28%),
ash level: 4.7 g/L (8%).

The active ingredient obtained is composed of molecules with sizes of less than 2 KDa.

### Example 7—Composition according to the invention

An example of a formulation comprising the active ingredient according to the invention in the form of anti-aging gel-cream characterized by a fresh and smooth texture is shown in table 1 below.

TABLE 1

| | Ingredients | % |
|---|---|---|
| A1 | Water | q.s. 100 |
| A2 | Butylene Glycol | 3.00 |
| | Sclerotium Gum & Xanthan Gum | 0.20 |
| B | Dimethicone | 8.00 |
| | Dimethicone | 8.00 |
| | Caprylyl Methicone | 4.00 |
| | Polysorbate 80 | 2.00 |
| | Hydroxyethyl Acrylate/Sodium Acryloyldimethyl Taurate | 0.87 |
| | Copolymer | |
| C | Hydroxyethyl Acrylate/Sodium Acryloyldimethyl Taurate | 2.00 |
| | Copolymer & Squalane & Polysorbate 60 | |
| D | Active ingredient according to the invention | 2.50 |
| E | Preservative | qs |

The composition of example 7 can in particular be obtained by the following method:
a. Add A2 in A1 under stirring and homogenize for 20 minutes.
b. Add B and then C into A under vigorous stirring. Stir for 20 minutes.
c. Add D and E with moderate stirring.

The composition is then in the form of a thick and glossy off-white gel-cream.

### Example 8—Composition according to the invention

An example of a formulation comprising the active ingredient according to the invention in cream form is presented in table 2 below.

TABLE 2

| | Ingredients | % |
|---|---|---|
| A1 | Water | q.s. 100 |
| | Sodium Gluconate | 0.20 |
| | Sodium Benzoate | 0.30 |
| A2 | Pentylene Glycol | 5.00 |
| | Sclerotium Gum & Xanthan Gum | 1.00 |
| B | Sodium Stearoyl Glutamate | 0.50 |
| | Cetearyl Alcohol & Cetearyl Glucoside | 3.00 |
| | Cera Alba (Beeswax) | 0.50 |
| | Astrocaryum Murumuru Seed Butter | 1.50 |
| | Hydrogenated Vegetable Oil | 0.75 |
| | Coco-Caprylate/Caprate | 6.00 |
| | Triheptanoin | 6.50 |
| | Caprylic/Capric Triglyceride | 8.25 |
| | Helianthus Annuus (Sunflower) Seed Oil | 4.00 |
| | Tocopherol & Helianthus Annuus (Sunflower) Seed Oil | 0.10 |
| C | Active ingredient according to the invention | 2.50 |
| D | Citric acid solution 10% | qs pH |

The composition of example 8 can in particular be obtained by the following method:
a. Add A2 into A1 under moderate stirring, stir until a homogeneous gel is obtained and heat to 80° C.,
b. Place B under magnetic stirring and heat to 80° C.,
c. Emulsify B in A under shearing stirring for 10 minutes,
d. At 30° C., under moderate stirring, add C, and
e. Adjust pH to 5.0-5.5 with D.

The composition is then in the form of a supple and shiny, broken white cream.

### Example 9—Compositions according to the invention

An example of a formulation comprising the active ingredient according to the invention in hybrid cream form, half care, half foundation, is presented in table 3 below.

TABLE 3

| | Ingredients | % |
|---|---|---|
| A1 | Water | q.s. 100 |
| | Preservative | qs |
| A2 | Glycerin | 1.50 |
| | Sclerotium Gum & Xanthan Gum | 0.10 |
| B1 | PEG-100 Stearate & Glyceryl Stearate | 5.00 |
| | Cetearyl Alcohol | 1.50 |
| | Triheptanoin | 3.50 |
| | Cetearyl ethylhexanoate | 3.00 |
| | Isodecyl Neopentanoate | 4.00 |
| | C12-15 alkyl benzoate | 3.00 |
| | Dimethicone | 0.50 |
| B2 | Ethylhexyl Methoxycinnamate | 3.00 |
| | Ethylhexyl Salicylate | 3.00 |
| C | Polyacrylate-13 & Polyisobutene & Polysorbate 20 | 0.50 |
| D | Polyacrylate Crosspolymer-6 | 0.50 |
| E | Butylene Glycol | 2.00 |
| | Alumina & CI 77891 (Titanium Dioxide) | 1.50 |
| | Alumina & CI 77891 (Titanium Dioxide) | 1.50 |
| F | Active ingredient according to the invention | 2.50 |
| G | Butylene Glycol | 2.00 |
| | Titanium Dioxide & Mannitol & CI 77492 (Iron Oxides) | 3.00 |
| | & Hydrogenated Lecithin & Polymethyl Methacrylate & | |
| | CI 77491 (Iron Oxides) & Aluminum Hydroxide & CI | |
| | 77499 (Iron Oxides) | |

The composition of example 9 can in particular be obtained by the following method:
a. Add A2 in A1 under stirring, stir until homogeneous and heat to 80° C.,
b. Place B1 under stirring and heat to 80° C. Add B2 in B1 just before emulsification,
c. Emulsify B in A under shearing stirring for 10 minutes,
d. At 50° C., under moderate stirring, add C then D, E and F.
e. At room temperature, add G under very gentle stirring.

The composition is then in the form of a supple, shiny, pale gray emulsion with gray particles in suspension.

### Example 10—Composition according to the invention

An example of a formulation comprising the active ingredient according to the invention in regenerating night cream form is presented in table 4 below.

TABLE 4

| | Ingredients | % |
|---|---|---|
| A1 | Water | q.s. 100 |
| | Preservative | qs |
| | Sorbitol 70% 1 | 1.00 |
| A2 | Propylene glycol | 3.00 |
| | Xanthan Gum | 0.30 |
| B | Glyceryl Stearate Citrate & Sucrose Stearate & | 6.00 |
| | Polyglyceryl-4 Cocoate & Cetyl Alcohol & | |
| | Sodium Ricinoleate | |
| | Hydrogenated Rapeseed Oil | 2.00 |
| | Cetearyl ethylhexanoate | 3.50 |
| | Octyldodecanol | 3.50 |
| | Squalane | 3.50 |
| | Pentaerythrityl Tetraisostearate | 3.50 |
| | Diisopropyl Adipate | 3.50 |
| | Dimethicone | 1.50 |
| | Argania Spinosa Kernel Oil | 2.00 |
| | Tocopherol & Helianthus Annuus (Sunflower) Seed Oil | 0.05 |
| C | Hydroxyethyl Acrylate/Sodium Acryloyldimethyl Taurate | 1.00 |
| | Copolymer & Polyisobutene & PEG-7 Trimethylolpropane | |
| | Coconut Ether | |
| D | Active ingredient according to the invention | 2.50 |

The composition of example 10 can in particular be obtained by the following method:
a. Add A2 into A1 under moderate stirring, stir until a homogeneous gel is obtained and heat to 80° C.,
b. Place B under magnetic stirring and heat to 80° C.,
c. Emulsify B in A under shearing stirring for 10 minutes, and
d. At 50° C., under moderate stirring, add C then D.

The composition is then in the form of a supple, shiny, pale beige emulsion. At 1 month, the pH is equal to 5.4 and the viscosity is equal to (C/5 rpm) 57400 cP.

### Evaluation of the Cosmetic Effect of the Active Ingredient According to the Invention

### Test 1—Effect of the Active Ingredient According to the Invention on its Capacity to Restore the Regenerating Fibroblast Complex.

The objective of this test is to evaluate the impact of aging on the expression and synthesis of growth factors: TGF-1, KGF, VEGF, FGF2, EGF, IGF1 and PDGFA present within the regenerating fibroblast complex.

For this study, the following parameters were analyzed on human fibroblasts derived from young and old facial tissue.

The expression of EGF, IGF1 and PDGFA was evaluated by QPCR and the synthesis of TGF-31, KGF, VEGF and FGF2 was evaluated by ELISA assay.

The procedure of the study is described below.

The young and older human fibroblasts are seeded and incubated at 37° C. in an atmosphere containing 5% CO₂.

The cells are then treated with the extracts at 0.25% and 1.00% (V/V). The cells are then incubated at 37° C. in an atmosphere containing 5% CO₂.

Finally, the fibroblasts are recovered and the total RNAs extracted.

The RNAs were reverse-transcribed and the complementary DNAs obtained were analyzed by the quantitative PCR technique. The internal reference control mRNAs were analyzed in parallel with the mRNAs of EGF, IGF1 and PDGFA.

The incorporation of fluorescence (SYBR Green) is measured continuously using a thermal cycler. The RQ (relative quantification) analysis is performed using software.

The supernatants are recovered and stored at −80° C. while awaiting ELISA assays of TGFβ1, KGF, VEGF, FGF2.

The objective of this study is to evaluate the effect of the examples according to the invention on the expression of growth factors: EGF, IGF1 and PDGFA of the regenerating fibroblast complex.

The results are shown in Table 5.

TABLE 5

| | | |
|---|---|---|
| Ability to | Ability to | Ability to |
| restore | restore | restore |
| EGF | IGF1 | PDGFA |
| expression | expression | expression |
| (%) | (%) | (%) |

Young Fibroblasts

Control

Older fibroblasts

| | | | |
|---|---|---|---|
| Control | −44 | −42 | −41 |
| EXAMPLE 2 (HI) | — | 4 | — |
| 1.00% | | | |
| INVENTION | +54 | +50 | +49 |
| 0.25% | | | |
| INVENTION | +98 | +75 | +76 |
| 1.00% | | | |

In comparison to young fibroblasts, the older fibroblasts have a reduced capacity to express: EGF by −44%; IGF-1 by −42%; PDGFA by −41%.

Aging leads to a significant depletion of the growth factors of the regenerating fibroblast complex.

The active ingredient according to the invention, for its part, significantly restores the expression of growth factors: EGF, IGF1 and PDGFA.

This test was also carried out in order to evaluate the effect of the active ingredient according to the invention on the synthesis of growth factors: TGFβ1, VEGF, KGF and FGF2 of the regenerating fibroblast complex. The results are shown in Table 6.

TABLE 6

| | | | |
|---|---|---|---|
| Ability to | Ability to | Ability to | Ability to |
| restore | restore | restore | restore |
| TGFβ1 | VEGF | KGF | FGF2 |
| synthesis | synthesis | synthesis | synthesis |
| (%) | (%) | (%) | (%) |

Fibroblasts

Control

Older fibroblasts

| | | | | |
|---|---|---|---|---|
| Control | −19 | −36 | −44 | −29 |
| EXAMPLE 2 (HI) 0.50% | — | — | 0 | — |
| Example 2 (HI) 2.00% | — | — | 0 | 0 |
| Example 3 (HI) 2.00% | — | — | — | 0 |
| INVENTION 0.25% | +85 | +50 | +25 | +26 |
| INVENTION 1.00% | +135 | +73 | +48 | +49 |
| EXAMPLE 4 (HI) | +6 | 0 | — | — |
| EXAMPLE 5 (HI) | 0 | 0 | — | — |
| EXAMPLE 6 (HI) | 0 | 0 | — | — |

In comparison to young fibroblasts, the older fibroblasts have a reduced capacity to synthesize: TGF-31 by −19%; VEGF by −36%; KGF by −44%; FGF2 by −29%.

Aging leads to a significant depletion of the growth factors of the regenerating fibroblast complex.

The active ingredient according to the invention significantly restores the synthesis of the growth factors, while the examples outside the invention do not have an effect on all the growth factors studied.

Tested at 1% on older human fibroblasts, the active ingredient according to the invention significantly restores the expression or the synthesis of growth factors: EGF by 98%, IGF1 by 75%, PDGFA by 76%, TGFβ1 by 135%, KGF by 48%, VEGF by 73%, and FGF2 by 49%.

The active ingredient according to the invention thus makes it possible to enrich the regenerating fibroblast complex of the older cells in growth factors.

### Test 2—Effect of the Active Ingredient According to the Invention on Fibroblast Migration.

The objective of this study is to evaluate the effect of the active ingredient according to the invention on the migration of older human fibroblasts.

In response to mechanical stress, the cells move within the tissue so as to locate themselves at the pressure area. In this area, contraction forces are then generated in order to maintain the integrity of the skin. With age, the migration capacity of the cells is reduced.

This study was carried out by a “time lapse” cell migration test on human fibroblasts derived from young and older facial tissue.

The effect is measured by evaluating the capacity of the cells to recolonize an injured area.

The young and older human fibroblasts are seeded and incubated at 37° C. in an atmosphere containing 5% CO₂.

The injured area is carried out after four days. The cells are treated with the extracts at 0.25% and/or 1.00% and/or 2% (V/V). They are then incubated in an atmosphere containing 5% CO₂ at 37° C.

The visualization of the fibroblast migration is carried out in time lapse owing to a fully automated system, which makes it possible to monitor the migration of the cells during the culture.

The migration rate of the cells within the injury is calculated owing to an internally developed image analysis script. The results are expressed in μm²/min.

The results are shown in Table 7.

TABLE 7

| | Capacity to restore |
|---|---|
| | migration rate (%) |
| Control (/young fibroblasts) | −27 |
| INVENTION 0.25% (/control) | +54 |
| INVENTION 1.00% (/control) | +97 |
| Example 2 (HI) 1% (/control) | +14 |
| Example 2 (HI) 2% (/control) | Negative |

The migration rate of the older human fibroblasts is significantly reduced by 27% relative to the young human fibroblasts.

Tested at 1.0% on older human fibroblasts, the active ingredient according to the invention significantly restores the migration rate by 97%. This is contrary to example 2, outside the invention, which has a slow migration rate.

The active ingredient according to the invention thus promotes faster recolonization of the older injured dermis, which is a key step of skin regeneration.

### Test 3—Effect of the Active Ingredient According to the Invention on Myofibroblast Differentiation.

The objective of this study is to evaluate the effect of the active ingredient according to the invention on the synthesis of alpha-Smooth muscle actin (α-SMA), actin microfilaments characteristic of myofibroblasts, in an injured area.

In the injured tissues, the fibroblasts are activated and differentiate into myofibroblasts, which contract and participate in healing by reducing the size of the wound and by secreting extracellular matrix proteins.

The study was carried out by immunocytofluorescence, on human fibroblasts derived from young and older facial tissue.

The procedure of the study is described below. The young and older human fibroblasts are seeded and incubated at 37° C. in an atmosphere containing 5% CO₂ and cultivated for several days.

The injury is carried out. The cells are treated with the active ingredient according to the invention at 0.25% and 1.00% (V/V) for 72 h. They are then incubated in an atmosphere containing 5% CO₂ at 37° C.

Immunocytological labeling of α-SMA is carried out by fixing and permeabilization using a primary antibody and a secondary antibody coupled to a fluorophore.

The visualization is performed using a microscope coupled to a camera and an image analysis system.

The intensity of the α-SMA labeling is proportional to the intensity of coloration (fluorescence) present in the cytoplasm of the fibroblasts. The greater the fluorescence, the higher the α-SMA synthesis rate.

A quantitative analysis of the images was carried out using an image analysis script developed internally. The results are expressed in arbitrary units (AU).

The results are shown in Table 8.

TABLE 8

| | |
|---|---|
| Synthesis of α-SMA | Ability to stimulate α- |
| (×10² AU) | SMA synthesis (%) |

Fibroblasts

| | |
|---|---|
| Control | 832 |

Older fibroblasts

| | | |
|---|---|---|
| Control | 205 | −75% |
| INVENTION 0.25% | 242 | +18% |
| INVENTION 1.00% | 286 | +40% |

The synthesis of α-SMA, a characteristic marker of myofibroblasts, is significantly reduced by 75% in an injured model in older cells compared to young cells.

When tested at 1% on older fibroblasts, the active ingredient according to the invention significantly stimulated synthesis of α-SMA by 40%.

The active ingredient according to the invention thus makes it possible to transform fibroblasts into myofibroblasts, which are key players in dermal regeneration.

### Test 4—Effect of the Active Ingredient According to the Invention on Keratinocyte Migration.

The objective of this study is to evaluate the impact of the older regenerating fibroblast complex that is treated with the active ingredient according to the invention on the migration of older human keratinocytes.

In response to mechanical stress, the cells move within the tissue so as to locate themselves at the pressure area. In this area, contraction forces are then generated in order to maintain the integrity of the skin. With age, the migration capacity of the cells is reduced.

This study was carried out by a time lapse cell migration test on human keratinocytes from young and older donors. The effect is measured by evaluating the capacity of the cells to recolonize an injured area.

The young and older human keratinocytes are seeded and incubated at 37° C. in an atmosphere containing 5% CO₂.

The injury is carried out. The cells are treated with secretomes from young fibroblasts (SFJ) or older fibroblasts (SFA) in the presence or absence of the active ingredient according to the invention at 0.25% and 1.00% (V/V). The cells are then incubated in an atmosphere containing 5% CO₂ at 37° C.

The visualization of the keratinocyte migration is carried out in time lapse owing to a fully automated system, which makes it possible to monitor the migration of the cells during the culture.

The migration rate of the cells within the injury is calculated owing to an internally developed image analysis script. The results are expressed in μm²/min.

The results are shown in Table 9.

TABLE 9

Ability to restore
migration rate (%)

Young keratinocytes

| | |
|---|---|
| SFJ Control | |
| SFJ INVENTION 1.00% | +13 |

Older keratinocytes

| | |
|---|---|
| SFA Control | −45 |
| SFA INVENTION 0.25% | +91 |
| SFA INVENTION 1.00% | +99 |
| SFA EXAMPLE 2 (HI) 1.00% | +39 |

The migration of the older human keratinocytes is significantly reduced by 45% compared to the young human keratinocytes.

Tested on older human keratinocytes, the secretome originating from older human fibroblasts pretreated with 1.0% of the active ingredient according to the invention significantly restores the cell migration rate by 99%. Conversely, in the presence of example 2 outside the invention, the cell migration rate is only restored by 39%.

The active ingredient according to the invention thus accelerates keratinocyte migration, which is a key step of skin regeneration.

### Test 5—Effect of the Active Ingredient According to the Invention on the Immune System and Macrophage Polarization.

The objective of this study is to evaluate the impact of the older regenerating fibroblast complex that is treated with the active ingredient according to the invention on the polarization of pro-regenerating macrophages.

The immune system is involved in defenses against pathogens, but also participates in tissue homeostasis. During healing, the regeneration phase is in particular guided by a population of pro-regenerating macrophages to the anti-inflammatory action and which are capable, inter alia, of stimulating matrix remodeling. With age, the activity of these macrophages decreases, in particular with less strong reactivity to external stimuli, resulting in less good differentiation and a decrease in expression of certain surface receptors necessary for their functionality.

This study was carried out on human macrophages derived from monocytes extracted from human peripheral blood. The polarization in pro-regenerating macrophages was evaluated by using flow cytometry to analyze the synthesis of the CD163 marker specific for this population and contributing to the anti-inflammatory response. In order to ensure that no population of pro-inflammatory macrophages was induced by the treatments, the synthesis of HLA-DR, a preferred marker of this population, was analyzed by flow cytometry and was complemented by the analysis of TNFα synthesis by an ELISA assay.

The procedure of the study is described below. The human monocytes are isolated from peripheral blood mononuclear cells (PBMCs) and are seeded in a macrophage differentiation medium before being incubated at 37° C. in an atmosphere containing 5% CO₂.

The macrophages are then treated with secretomes from young fibroblasts (SFJ) or older fibroblasts (SFA) in the presence or absence of the active ingredient according to the invention at 0.25% and 1.00% (V/V). The cells are then incubated in an atmosphere containing 5% CO₂ at 37° C.

The macrophages are recovered and labeled with conjugated antibodies specific to CD163 and HLA-DR. The cells are then fixed in a 1% paraformaldehyde solution before being analyzed on a flow cytometer equipped with analysis software.

The degree of synthesis of CD163 and HLA-DR is evaluated via the fluorescence median associated with each of these markers and is expressed in arbitrary units (AU).

In parallel, the supernatants are removed and stored at −80° C. until the analysis by ELISA assay of the TNFα.

The results are shown in Table 10.

TABLE 10

| | Pro-regenerating macrophages | |
|---|---|---|
| | Synthesis of | Ability to restore |
| | CD163 (AU) | CD163 synthesis (%) |
| SFJ Control | 231,811 | |
| SFA Control | 178,281 | −23% |
| SFA INVENTION 0.25% | 248,847 | +132 |

The secretome of young or older fibroblasts treated or not with the active ingredient according to the invention does not modify the HLA-DR synthesis rate and does not induce the synthesis of TNFα.

The secretome from human fibroblasts promotes the generation of pro-regenerating macrophages without inducing pro-inflammatory macrophages.

The degree of synthesis of CD163, specific receptor participating in the functionality of pro-regenerating macrophages, is reduced by 23% in the presence of secretome from older human fibroblasts compared to the secretome from young human fibroblasts.

The secretome from older human fibroblasts pretreated with 0.25% of the active ingredient according to the invention restores the surface expression of CD163 by 132%.

The active ingredient according to the invention thus reinforces the functionality of pro-regenerating macrophages, which are essential elements for skin regeneration.

### Test 6—Effect of the Active Ingredient According to the Invention on the Vascular System and the Migration of Endothelial Cells.

The objective of this study is to evaluate the effect of the active ingredient according to the invention on the migration of older human endothelial cells.

The endothelial cells composing the cutaneous micro-vessels are normally dormant in the capillaries. However, in response to a stimulus of the microenvironment (hypoxia, injury), endothelial cells are activated, migrate and are capable of remodeling the vascular network in order to meet the needs of the injured tissue. With age, senescent endothelial cells are dysfunctional, resulting in vascular remodeling defects as well as a decrease in the number of capillaries in the skin.

This study was carried out by a time lapse cell migration test, on young and older endothelial cells. The effect is measured by evaluating the capacity of the cells to recolonize an injured area.

The young and older endothelial cells (HUVEC: Human umbilical vein endothelial cells) are seeded and incubated at 37° C. in an atmosphere containing 5% CO₂.

The injury area is produced. The cells are treated with secretomes from young fibroblasts (SFJ) or older fibroblasts (SFA) in the presence or absence of the active ingredient according to the invention at 0.25% and 1.00% (V/V). The cells are then incubated in an atmosphere containing 5% CO₂ at 37° C.

The visualization of the endothelial cell migration is carried out in time lapse owing to a fully automated system, which makes it possible to monitor the migration of the cells during the culture.

The migration rate of the cells within the injury is calculated owing to an internally developed image analysis script. The results are expressed in μm²/min.

The results are shown in Table 11.

TABLE 11

| | |
|---|---|
| Migration | Ability to restore |
| rate (μm²/min) | migration rate (%) |

Young endothelial cells

| | |
|---|---|
| SFJ Control | 1009 |
| SFJ INVENTION 1.00% | 1021 |

Older endothelial cells

| | | |
|---|---|---|
| SFA Control | 532 | −47 |
| SFA INVENTION 0.25% | 759 | +48 |
| SFA INVENTION 1.00% | 975 | +93 |

The migration of the older human endothelial cells is significantly reduced by 47% relative to the young endothelial cells.

Tested on older human endothelial cells, the secretome originating from older human fibroblasts pretreated with 1.0% of the active ingredient according to the invention significantly restores the migration rate of the endothelial cells by 93%.

The active ingredient according to the invention thus accelerates the migration of the older endothelial cells, which is essential to the regeneration of the (injured) vascular network.

### Test 7—Effect of the Active Ingredient According to the Invention on the Vascular System and the Cohesion of Endothelial Cells.

The objective of this study is to evaluate the effect of the active ingredient according to the invention, within the regenerating fibroblast complex, on the synthesis of ZO-1 (Zonula Occludens-1), junction protein between the endothelial cells.

Vascular integrity is ensured by establishing adherent and tight junctions between endothelial cells. These junctions allow selective permeability of the vessels. ZO-1 is a constituent component of the tight endothelial junctions. With age, endothelial cells are dysfunctional and have a decrease in the expression and membrane addressing of the components of the tight junctions, inducing an increased permeability.

The study was carried out by immunocytofluorescence, on young and older endothelial cells.

The young and older human endothelial cells (HUVEC: Human umbilical vein endothelial cells) are seeded and incubated at 37° C. in an

The cells are treated with secretomes from young fibroblasts (SFJ) or older fibroblasts (SFA) in the presence or absence of the active ingredient according to the invention at 0.25% and 1.00% (V/V). They are then incubated in an atmosphere containing 5% CO₂ at 37° C.

Immunocytological labeling of ZO-1 is carried out by fixing and permeabilization using primary antibody and secondary antibody coupled to a fluorophore.

Viewing is performed using a microscope coupled to an image analysis system.

The fluorescence surface, corresponding to the ZO-1 labeling, is proportional to the establishment of tight junctions between the endothelial cells. A quantitative analysis of the images was carried out using an image analysis script in the Python language. The results are expressed in arbitrary units (AU).

The results are shown in Table 12.

TABLE 12

| | |
|---|---|
| Synthesis | Ability to restore |
| of ZO-1 (AU) | ZO-1 synthesis (%) |

Young endothelial cells

| | |
|---|---|
| SFJ Control | 6742 |
| SFJ INVENTION 1.00% | 7115 |

Older endothelial cells

| | | |
|---|---|---|
| SFA Control | 3232 | −52 |
| SFA INVENTION 0.25% | 5003 | +50 |
| SFA INVENTION 1.00% | 939 | +77 |

The synthesis of ZO-1, constituent protein of intercellular junctions, is significantly reduced by 52% in older endothelial cells compared to young cells.

Tested at 1.0% on older endothelial cells, the active ingredient according to the invention significantly restores the synthesis of ZO-1 by 77%.

The active ingredient according to the invention thus makes it possible to reinforce cell cohesion.

### Test 8—Effect of the Active Ingredient According to the Invention on the Vascular System and the Formation of the Vascular Network.

The objective of this study is to evaluate the effect of the active ingredient according to the invention on the formation of a capillary network of older endothelial cells.

The remodeling of the vascular skin network in adults in response to a stimulus (hypoxia, injury) is carried out via an angiogenesis mechanism. This process is defined as the formation of vessels from pre-existing vessels. It is the result of specification, migration and proliferation of endothelial cells. Physiological angiogenesis allows the skin to heal correctly. With age, the formation of capillaries by angiogenesis is altered and prevents good tissue healing and homeostasis.

The study was carried out by immunocytofluorescence, on young and older endothelial cells owing to the specific endothelial cell marker: PECAM-1 (CD31).

Young or older fibroblasts are seeded and incubated at 37° C. in an

The cells are treated with the 1.00% extracts (V/V) and incubated at 37° C. in an atmosphere containing 5% CO₂.

The young and older human endothelial cells (HUVEC: Human Umbilical Vein Endothelial Cells) are seeded on the young or older fibroblast mats and incubated at 37° C. in an atmosphere containing 5% CO₂.

The cells are then treated with an extract at 1.00% (V/V) and incubated at 37° C. in an atmosphere containing 5% CO₂.

Immunocytological labeling of CD31 is carried out by fixing and permeabilization using primary antibody and secondary antibody coupled to a fluorophore.

The visualization is performed using a microscope coupled to a camera and an image analysis system.

The fluorescence surface corresponding to the CD31 labeling is proportional to the extent of the vascular network formed by the endothelial cells.

A quantitative analysis of the images was carried out using an image analysis script developed internally. The results are expressed in arbitrary units (AU).

The results are shown in Table 13.

TABLE 13

| | Ability to restore the |
|---|---|
| | formation of a |
| | capillary network (%) |
| Young endothelial cells | |
| Control | |
| INVENTION 1.00% | +57 |
| EXAMPLE 3 1.00% | +28 |
| Older endothelial cells | |
| Control | |
| INVENTION 1.00% | +101 |
| EXAMPLE 3 1.00% | +77 |

Tested at 1.0% on older endothelial cells, the active ingredient according to the invention makes it possible to significantly restore the formation of a capillary network by 101%. Conversely, example 3 outside the invention has a lower efficacy.

The active ingredient according to the invention thus promotes angiogenesis.

### Test 9—Study of the Biological Activity of the Active Ingredient According to the Invention In Vivo.

The objective of this study is to evaluate in vivo the biological activity of the active ingredient according to the invention formulated at 2.5% in emulsion form, compared to a placebo formula, on Caucasian and Asian volunteers.

The Caucasian panel is composed of 19 healthy female volunteers, aged 70 to 78 years, having wrinkles of the crow's feet and a dull complexion.

The Asian panel consisted of 33 people aged 60 to 69 years and the placebo group of 32 people aged 60 to 68 years.

Effect of the active ingredient according to the invention on cell renewal.

Firstly, the effect of the active ingredient according to the invention was evaluated at the forearm on the Caucasian panel by studying its ability to accelerate cell renewal after 14 days of twice-daily application.

Cell renewal is measured owing to the capacity of the skin to eliminate DHA (dihydroxyacetone), which is a marker dying proteins. The cell renewal leads to the disappearance of the coloration caused by the DHA.

A gel containing 8% DHA is applied to the surface of the skin of the volunteers. The monitoring of the color obtained after application of the DHA gel starts 72 hours after staining on untreated areas, treated twice daily with a placebo formula or treated twice daily with a formula containing 5% of the active ingredient according to the invention. The evaluation is carried out from photographs of the skin taken at regular intervals for 14 days, using a dermatoscope.

The results corresponding to the effect of the active ingredient according to the invention formulated at 2.5% in emulsion form on the ability to eliminate DHA are presented in table 1.

In comparison to the placebo, the active ingredient according to the invention formulated at 2.5% emulsion promotes epidermal dynamics by increasing the skin's capacity to eliminate DHA.

This effect is observed and significant from 2 days after the application of the products and continues throughout the study (D14).

Indeed, with the active ingredient according to the invention, in less than 7 days, the staining with DHA is removed at more than 80%, versus 11 days for the area treated with the placebo.

### Effect of the Active Ingredient According to the Invention on Dermis Structure.

Secondly, the effect of the active ingredient according to the invention on the structure of the dermis was evaluated at the face after 21 and 42 days of application by evaluating the quality of the matrix by LC-OCT (for Line-Field Optical Coherence Tomography). This study was carried out on Caucasian and Asian panels.

The results showing the effect on the Caucasian panel of the active ingredient according to the invention on the quality of the dermal matrix evaluated over a thickness of 50 μm under the DEJ are shown in table 14 below.

TABLE 14

| | D 21 | D 42 |
|---|---|---|
| Placebo | −2 | −1 |
| INVENTION 2.5% | 30 | 27 |

From 21 days of treatment, the active ingredient according to the invention formulated at 2.5% significantly improves the quality of the matrix of Caucasian volunteers by 32% relative to the placebo.

This effect was observed in 89% of the subjects. Thus, the matrix dynamics of the dermis are restored. This effect lingers after 42 days of treatment.

The results showing the effect of the active ingredient according to the invention on the quality of the dermal fibers of the Asian volunteers are shown in table 15 below.

TABLE 15

| | D 21 | D 42 |
|---|---|---|
| Placebo | 1 | 0 |
| INVENTION 2.5% | 21 | 18 |

From 21 days of treatment, the active ingredient according to the invention formulated at 2.5% significantly improves the quality of the fibers of Asian volunteers by 20% relative to the placebo. This effect was observed in 97% of the Asian subjects. This effect lingers after 42 days of treatment.

### Test 10—Evaluation of the Cosmetic Benefits of the Active Ingredient According to the Invention

The objective of this study is to evaluate in vivo the cosmetic benefits of the active ingredient according to the invention formulated at 2.5% in emulsion form, compared to a placebo formula, on Caucasian and Asian volunteers. The effect of the active ingredient according to the invention was evaluated after 21 and 42 days of twice-daily application.

The Caucasian panel is composed of 19 healthy female volunteers, aged 70 to 78 years, having wrinkles of the crow's feet and a dull complexion.

The Asian panel consists of 33 people aged 60 to 69 years and the placebo group of 32 people aged 60 to 68 years.

### Action on the Biomechanical Properties of the Skin on Caucasian Panel

The results corresponding to the effect of the active ingredient according to the invention on the biomechanical properties of the skin of Caucasian volunteers are shown in FIG. 2.

The active ingredient according to the invention, compared to the placebo, improves the biomechanical properties of the skin of Caucasian volunteers.

Indeed, from 21 days of treatment, the active ingredient according to the invention increases by:
10% for the firmness of the skin, effect observed in 84% of subjects
14% for the skin elasticity, observed in 68% of subjects
25% for the tonicity of the skin, effect observed in 84% of subjects.

In addition, 79% of volunteers find their skin to be firmer and 79% find it more tonic with the active ingredient according to the invention, versus 68% and 74%, respectively, for the placebo. This effect lingers after 42 days of twice-daily application.

### Anti-Wrinkle Action on Caucasian Panel

The results corresponding to the anti-wrinkle effect of the active ingredient according to the invention evaluated on the crow's feet area for

Caucasian volunteers by visual scoring on digital photographs compared to the placebo are shown in table 16.

TABLE 16

| | D 21 | D 42 |
|---|---|---|
| Placebo | 0 | 2 |
| INVENTION 2.5% | −10 | −8 |

From 21 days of treatment, compared to the placebo, the active ingredient according to the invention formulated at 2.5% by emulsion has an anti-wrinkle effect, by significantly reducing the stage of wrinkles of the crow's feet by 10%. This effect is observed in 74% of the volunteers.

In addition, 74% of subjects observed that their wrinkles and fine lines were less visible with the active ingredient according to the invention versus only 58% for the placebo treatment. This effect lingers after 42 days of treatment.

### Action on the Cutaneous Microvascular Network of the Caucasian Panel

The results corresponding to the effect of the active ingredient according to the invention on the visibility of the micro-vessels of the skin of

Caucasian volunteers are shown in table 17.

TABLE 17

| | D 21 | D 42 |
|---|---|---|
| Placebo | −5 | −14 |
| INVENTION 2.5% | −10 | −21 |

From 21 days of treatment and compared to the placebo, the active ingredient according to the invention formulated at 2.5% reduces the visibility of the dystrophic vessels at the cheeks (−6%).

This effect lingers after 42 days of treatment, where a significant reduction of 7% is observed; effect validated on 63% of subjects.

By decreasing the visibility of the dystrophic micro-vessels, the active ingredient according to the invention improves cutaneous vascularization.

### Action on Under-Eye Circles in Caucasian Panel

The results corresponding to the effect of the active ingredient according to the invention on under-eye circles in Caucasian volunteers are shown in table 18 below.

TABLE 18

| | D 21 | D 42 |
|---|---|---|
| Placebo | −0.3 | 1.6 |
| INVENTION 2.5% | −3.1 | −1.6 |

In comparison to the placebo, the active ingredient according to the invention formulated at 2.5% as an emulsion limits under-eye circles by decreasing parameter b* by 2.9% from 21 days of application and lingers until the end of the study. Parameter b* represents the blue to yellow range during colorimetric measurements; it is characteristic of cutaneous melanic yellow pigmentation. The more this parameter decreases, the more the skin will brighten.

This effect is visible in 68% of the volunteers, and 79% of them perceived the product as a concealer compared to only 53% for the placebo treatment.

### Action on the Radiance of the Complexion of the Caucasian Panel

The results corresponding to the effect of the active ingredient according to the invention formulated at 2.5% on the main parameters characteristic of the radiance of the complexion, evaluated by experts on the Caucasian panel, are shown in table 19 below.

TABLE 19

| | D 21 | D 42 |
|---|---|---|
| Radiance | 9 | 10 |
| Pinkness | 15 | 16 |
| Olive color | −7 | −11 |
| Eye fatigue | −6 | −8 |

From 21 days of twice-daily application, and in comparison to placebo, the active ingredient according to the invention, formulated at 2.5% in emulsion:
gives a lighter and fresher complexion by significantly increasing skin radiance by 9% and pinkness by 15%. Effect observed in 74% and 84% of the volunteers, respectively.
significantly reduces olive color by 7% and eye fatigue by 6%. This effect was observed in 79% and 89% of the volunteers, respectively.

This effect is reinforced by the evaluation of the volunteers: 84% on D21 were found to have more radiant skin with the active ingredient according to the invention versus 63% for the placebo treatment.

### Anti-Wrinkle Action on Asian Panel

The results corresponding to the anti-wrinkle effect of the active ingredient according to the invention studied by visual scoring by experts on Asian volunteers are shown in table 20. A panel composed of 3 people evaluated the stage of wrinkles of the crow's feet in each photo on a previously defined score scale.

TABLE 20

| | D 21 | D 42 |
|---|---|---|
| Placebo | 1 | −1 |
| INVENTION 2.5% | −5 | −7 |

From 21 days of treatment, compared to the placebo, the active ingredient according to the invention formulated at 2.5% by emulsion has an anti-wrinkle effect in Asian volunteers, by significantly reducing the stage of wrinkles of the crow's feet by 6%. This effect is observed in 58% of the volunteers. This effect lingers after 42 days of treatment.

### CONCLUSION

The active ingredient according to the invention formulated at 2.5% as an emulsion provides cosmetic benefits from 21 days of treatment, which linger after 42 days of treatment.

Indeed, the active ingredient according to the invention allows:
improved biomechanical properties of the skin
reduced wrinkles in Caucasian and Asian volunteers
improved skin vascularization, by reducing the dystrophic microvessels as well as under-eye circles
improved complexion radiance.

1. An active ingredient comprising at least one extract of Saccharomyces cerevisiae, characterized in that said extract corresponds to the cytosolic fraction comprising oligo-glucans with a molar mass of between 2 and 5 kDa.

2. The cosmetic active ingredient according to claim 1, characterized in that the oligo-glucans represent between 10 and 35 wt. % dry matter of the extract.

3. The cosmetic active ingredient according to claim 2, characterized in that the extract comprises a glucose/mannose ratio greater than 60%.

4. The cosmetic active ingredient according to claim 1, characterized in that the extract is a mechanical lysate of Saccharomyces cerevisiae.

5. The cosmetic active ingredient according to claim 1, characterized in that said extract is capable of being obtained by a method comprising the following steps:
a. solubilizing at least 50 g/l of Saccharomyces cerevisiae in water, by mechanical homogenization,
b. separating the soluble and insoluble phases,
c. ultrafiltration and recovery of the filtrate, and
d. molecular sorting.

6. A cosmetic composition for topical application comprising at least one active ingredient according to claim 1 and a physiologically acceptable medium.

7. The cosmetic composition according to claim 6, wherein the active ingredient in liquid form represents at least 0.1 wt. % of the total weight of the composition.

8. A cosmetic use of an active ingredient according to claim 1 or a cosmetic composition comprising the active ingredient, for improving skin regeneration related to normal aging of the skin.

9. The cosmetic use according to claim 8, characterized in that the extract or composition improves the interconnectivity of the skin, vascular and immune systems.

10. The cosmetic use according to claim 9, characterized in that the extract or the composition increases the synthesis and the secretion of KGF, EGF, IGF, FGF, PDGF, TGF-β and VEGF.

11. The cosmetic use according to claim 8 for improving the radiance of the complexion.

12. The cosmetic use according to claim 8 for preventing and/or reducing under-eye circles.

13. The cosmetic use according to claim 8 for preventing and/or reducing wrinkles.
